(19) United States
(12) Patent Application Publication
(10) Pub. No.: US 2022/0262505 A1
(43) Pub. Date: Aug. 18, 2022

(54) DRUG DELIVERY SYSTEM WITH ADJUSTABLE INJECTION TIME AND METHOD OF USE

(71) Applicant: AMGEN INC., Thousands Oaks, CA (US)

(72) Inventors: Azita Rahbari, Westlake Village, CA (US); Sunitha Dasoju, Ventura, CA (US); Antonio Antonini, Milano (IT); NIcola Antonio Alagia, Milano (IT); Ferdinando Valenti, Thousand Oaks, CA (US)

(21) Appl. No.: 17/626,688

(22) PCT Filed: Jul. 16, 2020
(86) PCT No.: PCT/US20/42222

§ 371 Date: Jan. 12, 2022

Related U.S. Application Data
(60) Provisional application No. 62/875,716, filed on Jul. 18, 2019.

(51) Int. Cl. G16H 40/63; A61M 5/20; A61M 5/24; G16H 10/60

(52) U.S. Cl. CPC G16H 40/63; A61M 5/20; A61M 2005/3126; G16H 10/60; A61M 2005/2496; A61M 5/24

(57) ABSTRACT

Drug delivery devices are configured to inject a drug over an injection time within an acceptable range calculated based on information pertaining to the drug to be injected. In particular, drug delivery devices are described that allow identification of the drug to be injected, calculation or determination of an acceptable range of injection times for the identified drug, determination of a tailored injection time within the acceptable range of injection times, and setting of a delivery speed of a drive such that the drug is expelled over the tailored injection time.

### CROSS-REFERENCE TO RELATED APPLICATION

This application claims the benefit of U.S. Provisional Application No. 62/875,716, filed on Jul. 18, 2019, which is hereby incorporated by reference herein in its entirety.

### FIELD OF THE DISCLOSURE

The present disclosure generally relates to a drug delivery system and, in particular, to a drug delivery system allowing a user to adjust an injection time within an acceptable range calculated based on drug information.

### BACKGROUND

Pre-filled hypodermic syringes provide several advantages for the home-use market. These advantages include that pre-filled syringes may be prepared for each medicament with more accurate dosages. Further, they are more easily operated, by merely advancing the stopper of the syringe. Aside from the costs of the particular medication used, pre-filled syringes can also be more economical to manufacture. Consequently, all these advantages make pre-filled syringes more commercially appealing.

Nevertheless, pre-filled syringes also have some significant drawbacks in the marketplace. Specifically, some users can be either frightened by an exposed needle or feel they are inherently incapable of performing an injection. Because of aversions to exposed needles, as well as health and safety issues that may be involved, various types of injectors and other devices have been developed for the specific purpose of concealing needles from the user and automating the injection task to assist the user in performing the injection.

In particular, automated drug delivery devices control the speed of drug drive components in order to accurately deliver a full dose of a drug in a pre-determined injection time. A wide variety of drugs are delivered to patients via drug delivery devices, and the characteristics of the drug being delivered, such as the viscosity and dose volume, influence the injection time. For some drugs, a maximum injection time and/or a minimum injection time is targeted in order to reduce injection pain or improve drug efficacy. Patients may have personal preferences regarding injection time. For example, some patients may prefer a faster injection time in order to complete drug delivery quickly. Other patients may find that a slower injection time is more comfortable.

### SUMMARY

Some aspects of the present disclosure include a drug delivery system including a reservoir, an identifier, a drug delivery device, a reader, a drive, and a controller. The reservoir is adapted to contain a drug. The identifier has drug information. The drug delivery device is adapted to receive the reservoir. The reader of the drug delivery device is adapted to read the drug information. The drive of the drug delivery device is adapted to expel the drug from the reservoir. The controller is coupled to the reader and the drive. The controller is programmed to (a) identify the drug contained in the reservoir based on the drug information, (b) calculate or determine an acceptable range of injection times for the identified drug, (c) determine a tailored injection time within the acceptable range of injection times, and (d) set a speed of the drive such that the drug is expelled from the reservoir over the tailored injection time.

In some forms, the controller calculates or determines the acceptable range of injection times at least in part based on the drug information.

In some forms, the drug information comprises at least one of viscosity, dose volume, minimum injection time to achieve drug efficacy, maximum injection time to achieve drug efficacy, minimum injection time to curtail injection pain, and maximum injection time to curtail injection pain.

In some forms, the controller includes a memory containing a drug information dataset having a list of possible drugs, each possible drug associated with drug information, and calculating or determining the acceptable range of injection times for the identified drug includes accessing the drug information dataset, determining that the identified drug is one possible drug in the list of possible drugs, and receiving the drug information associated with the identified drug in the drug information dataset.

In some forms, the drug delivery system includes an input device functionally coupled to the controller. The controller is further programmed to receive a preferred injection speed from the input device. The tailored injection time is based on the preferred injection speed.

In some forms, the input device includes two or more relative injection speed options, each relative injection speed option equal to a percentage of a longest possible injection time within the range of acceptable injection times, and wherein the preferred injection speed is the relative injection speed option selected using the input device.

In some forms, the input device includes a touchscreen, and wherein the controller causes the input device to display the two or more relative injection speed options.

In some forms, each of the two or more relative injection speed options are displayed as one of: a written adjective, the percentage of the longest possible injection, and a point on a sliding scale.

In some forms, the two or more relative injection speeds are each unique physical buttons of the drug delivery device.

In some forms, the identifier is an RFID tag or an NFC tag.

Other aspects of the present disclosure include a method of preparing a drug delivery device for delivering a drug product. The method can include identifying a drug contained in a reservoir of a drug delivery system based on a reader of the drug delivery system reading an identifier on the reservoir. The method can further include calculating an acceptable range of injection times for the identified drug. The method can further include receiving a preferred injection speed from an input device of the drug delivery system. The method can further include determining a tailored injection time within the acceptable range of injection times based on the preferred injection speed. The method can further include setting a speed of a drive of the drug delivery system such that the drug is expelled from the reservoir over the tailored injection time.

In some forms, calculating the acceptable range of injection times for the identified drug includes receiving drug information from the identifier on the reservoir.

In some forms, the drug information includes at least one of viscosity, dose volume, minimum injection time to achieve drug efficacy, maximum injection time to achieve drug efficacy, minimum injection time to curtail injection pain, and maximum injection time to curtail injection pain.

In some forms, calculating the acceptable range of injection times for the identified drug includes accessing a drug information dataset provided in a memory of the controller, determining that the identified drug is one possible drug in a list of possible drugs, and receiving drug information associated with the identified drug in the drug information dataset.

In some forms, the drug information includes at least one of viscosity, dose volume, minimum injection time to achieve drug efficacy, maximum injection time to achieve drug efficacy, minimum injection time to curtail injection pain, and maximum injection time to curtail injection pain.

In some forms, the method further comprises presenting two or more relative injection speed options, each relative injection speed option equal to a percentage of a longest possible injection time within the range of acceptable injection times, allowing selection of one of the two or more relative injection speed options, and using the relative injection speed option selected as the preferred injection speed.

In some forms, the method further comprises causing the input device to display the two or more relative injection speed options on a touchscreen.

In some forms, each of the two or more relative injection speed options are displayed as one of: a written adjective, the percentage of the longest possible injection, and a point on a sliding scale.

In some forms, allowing selection of one of the two or more relative injection speed options includes associating the two or more relative injection speeds with unique physical buttons of the drug delivery device.

In some forms, reading an identifier on the reservoir comprises reading an RFID tag or an NFC tag.

### BRIEF DESCRIPTION OF THE DRAWINGS

The above needs are at least partially met through provision of the embodiments described in the following detailed description, particularly when studied in conjunction with the drawings, wherein:

FIG. 1 is a schematic illustration of a drug delivery system including a drug delivery device in accordance with various embodiments of the invention.

FIG. 2 is an elevational side view of an exemplary embodiment of a drug delivery device comprising an autoinjector and a cassette.

FIG. 3A is a front elevational view of an exemplary embodiment of the autoinjector of FIG. 2.

FIG. 3B is an elevational view of a first side of the autoinjector of FIGS. 2 and 3A.

FIG. 3C is a rear elevational view of the autoinjector of FIGS. 2-3B.

FIG. 3D is an elevational view of a second side of the autoinjector of FIGS. 2-3C.

FIG. 3E is an elevational view of a first end of the autoinjector of FIGS. 2-3D.

FIG. 3F is an elevational view of a second end of the autoinjector of FIGS. 2-3F.

FIG. 3G is a sectional side view of the autoinjector apparatus of FIGS. 2-3F.

FIG. 4 is an exploded perspective view of an exemplary embodiment of the cassette of FIG. 2.

FIG. 5 is a flow chart illustrating the decision logic for injecting a drug using the autoinjector over a tailored injection time according to an exemplary embodiment of the present disclosure.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

A drug delivery system and method is provided that allows the injection time over which a drug is expelled to be tailored to suit a user's preferences within a range of acceptable injection times for the drug being injected. The patient benefits from this system by being able to adjust the injection time to suit personal preferences (i.e., minimizing total injection time, delivering the drug at a rate that is most comfortable) while still having the drug delivered in an injection time that is efficacious for the drug.

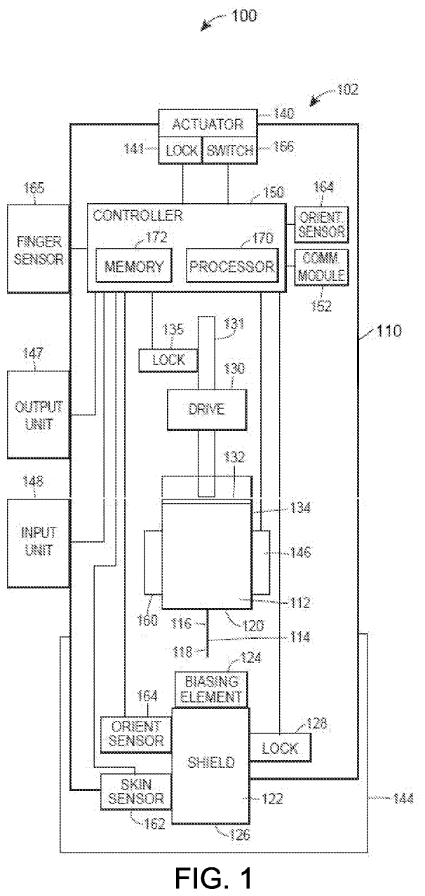

Referring now to the drawings, and in particular to FIG. 1, one generalized example of a system 100 is provided which includes a drug delivery device 102. The drug delivery device 102 may be in the form of an autoinjector, and thus is adapted for hand-held use and application against the skin of the patient. The drug delivery device 102 includes a housing 110 in which are disposed assemblies or structures that introduce a delivery cannula into the patient, and that eject a drug or medicament from a reservoir 112 through the delivery cannula into the patient. According to certain embodiments, the same assemblies or structures that introduce the delivery cannula into the patient may also eject the drug or medicament from the reservoir through the delivery cannula into the patient. The drug delivery device 102 may also include assemblies or structures that connect the delivery cannula to the reservoir, that withdraw the delivery cannula into the housing 110 through an opening in the housing 110 (not illustrated), or that deploy other structures that will prevent contact with the delivery cannula once the delivery cannula has been removed from the patient. Any number of additional assemblies and structures are possible. The specific embodiment of the drug delivery device 102 discussed below is thus by way of example and not by way of limitation.

Accordingly, the drug delivery device 102 includes a reservoir 112 (such as a reservoir provided in a cassette 200, discussed below) and a delivery cannula 114 having a first end 116 (e.g., a proximal end) that may be connected or connectable in fluid communication with the reservoir 112 and a second end 118 (e.g., a distal end) that may be inserted into a patient. The delivery cannula 114 may be, for example, a rigid needle having a beveled edge that may be sized such that the second end 118 of the needle 114 is received under the skin so as to deliver a subcutaneous injection of the medicament within the reservoir 112. The first end 116 of the needle 114 may be disposed through a wall 120 of the reservoir 112, and thus be connected in fluid communication with the reservoir 112. Alternatively, the first end 116 of the needle 114 may be disposed only partially through the wall 120 (which wall 120 may be a resalable septum or stopper, for example) such that the first end of the needle 114 may not be connected in fluid communication until the second end 118 of the needle 114 is inserted into the patient. In such a circumstance, the first end 116 of the needle 114 may thus be described as connectable in fluid communication with the reservoir 112, although it will be recognized that there are other mechanisms by which the first end 116 of the needle 114 may be connectable, but not connected, in fluid communication with the reservoir 112.

The drug delivery device 102 includes a shield 122 (e.g., a needle shield) that may be deployed at least after the injection has been completed to limit access to the second end 118 of the needle 114. According to certain embodiments, the shield 122 may have a biasing element 124 (such as a spring) that extends the shield 122 from the housing 110 such that a distal end 126 of the shield 122 extends beyond the second end 118 of the needle 114 except when the shield 122 is disposed against the skin and the insertion of the needle 114 is actuated. In fact, the insertion of the needle 114 may be actuated according to certain embodiments of the drug delivery device 102 by disposing the distal end 126 of the shield 122 on or against the skin of the patient.

The drug delivery device 102 may also include a lock 128 (e.g., a ratchet) that is coupled to the shield 122 and configured to limit or prevent movement of the shield 122 relative to the housing 110 of the drug delivery device 102 such that the distal end 126 of the shield 122 extends from the housing 110 a sufficient distance to limit or prevent contact with the second end 118 of the needle 114, for example, after the needle 114 has been removed or separated from the skin of the patient. In some embodiments, the lock 128 may be coupled to a controller (e.g., controller 150 described in more detail below) which can selectively activate or deactivate the lock 128 based on different types of information regarding the drug delivery device 102, including operational state information, condition information, and/or identity information. When the lock 128 is activated by the controller 150, the lock 128 may be configured to limit or prevent movement of the needle shield 122 relative to the housing 110. When the lock 128 is deactivated by the controller 150, the lock 128 may be configured to allow movement of the needle shield 122 relative to the housing 110.

The drug delivery device 102 also includes at least one drive 130 that may be used to insert the second end 118 of the needle 114 into the skin of the patient, and to eject the drug or medicament from the reservoir 112 through the delivery cannula 114 into the patient. The drive 130 may include a source of pressurized gas or a source of a material that undergoes a phase change, such that the escaping gas or phase changing material provides a motive force that may be applied to the reservoir 112 to eject the drug therefrom. According to other embodiments, the drive 130 may include an electromechanical system, such as may include a motor for example. Other embodiments of the drive 130 are also possible.

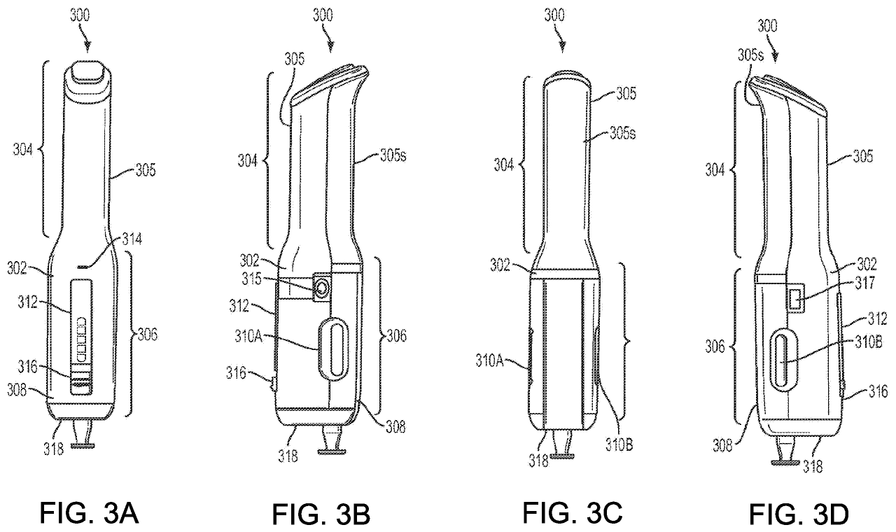

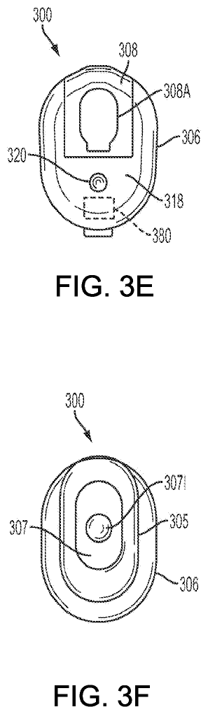

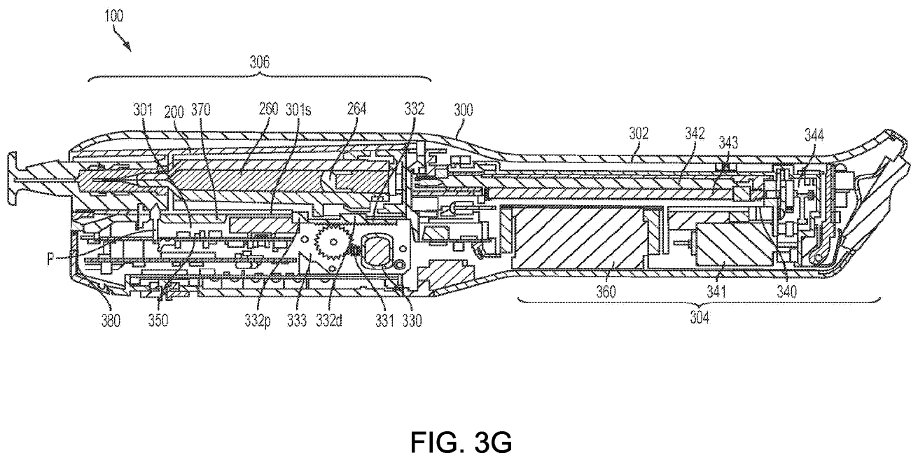

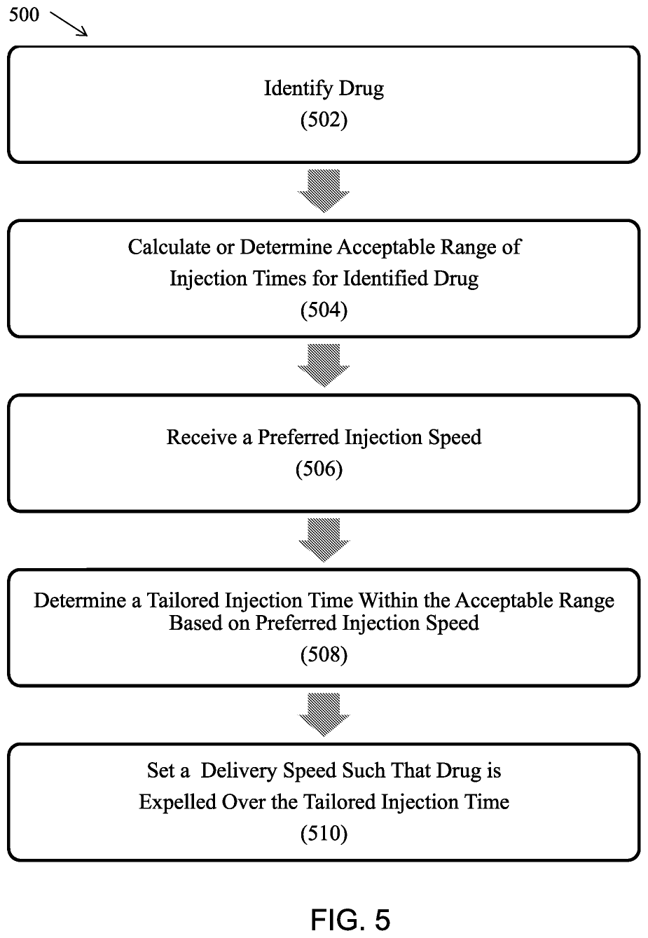

In one embodiment, the drive 130 includes a motor that is controlled by the controller 150, and the injection time over which a drug is extruded may be set by the controller 150. The decision logic implemented by the controller 150 and the drive 130 in order to set the injection time is described in greater detail in FIG. 5 below. In other embodiments, the same decision logic may be implemented by a controller 150 for a drive 130 that does not include a motor but instead relies upon other controllable force generating components (e.g., a pressurized gas system). The drive 130 may include the various components described below with respect to drive 340 of FIG. 3G.

In one embodiment, the drive 130 may be coupled to a plunger 131 and/or a stopper 132 (e.g., a wall) disposed in the reservoir 112 to move that stopper 132 in a distal direction toward the delivery cannula 114. In accordance with such an embodiment, the stopper 132 may be a stopper that is fixed to a distal end of the plunger 131 and received within a bore 134. The plunger 131, in conjunction with the drive 130, may move the stopper 132 along a longitudinal axis of the drug delivery device 102 through the bore 134 from a proximal end of the bore 134 to a distal end of the bore 134, and thereby eject the medicament from the reservoir 112.

In some embodiments, the drive 130 may also cooperate with the stopper 132 and/or the bore 134 to move the reservoir 112 relative to the housing 110 so as to move the second end 118 of the needle 114 relative to the housing 110 and into the patient. According to those embodiments wherein the drive 130 cooperates with the stopper 132, this may occur before the first end 116 of the needle 114 is in fluid communication with the reservoir 112. According to those embodiments wherein the drive cooperates with the bore 134, the drive may include one component (e.g., first spring) that cooperates with the bore 134 to move the reservoir 112 and needle 114 relative to the housing 110, and a second component (e.g., second spring) that cooperates with the stopper 132 to move the stopper 132 relative to the bore 134.

The drug delivery device 102 may also include a lock 135 that is coupled to the plunger 131 and configured to limit or prevent movement of the plunger 131 relative to the housing 110 of the drug delivery device 102 so that the stopper 132 cannot be advanced to discharge the medicament from the reservoir 112 to the patient. In some embodiments, the lock 135 may be coupled to a controller (e.g., controller 150 described in more detail below) which can selectively activate or deactivate the lock 135 based on different types of information regarding the drug delivery device 102, including operational state information, condition information, and/or identity information, in accordance with one or more of the methods described above. When the lock 135 is activated by the controller 150, the lock 135 may be configured to limit or prevent movement of the plunger 131 relative to the housing 110. When the lock 135 is deactivated by the controller 150, the lock 128 may be configured to allow movement of the plunger 131 relative to the housing 110.

The drive 130 may be associated with an actuator 140. The actuator 140 may activate the controller 150 to cause the drive 130 to insert the needle 114 and eject the drug from the reservoir 112 through the needle 114 into the patient. The actuator 140 may, according to certain embodiments, be the needle shield 122, as explained above. According to other embodiments, such as the one illustrated in FIG. 1, the actuator 140 may be a button that may be manually depressed by the user or patient once the drug delivery device 102 is placed disposed on or against the patient's skin. A lock 141 may be coupled to the actuator 140 and configured to limit or prevent movement of the actuator 140 so that the actuator 140 cannot be used to activate the drive 130. In some embodiments, the lock 141 may be coupled to a controller (e.g., controller 150) which can selectively activate or deactivate the lock 141 based on different types of information regarding the drug delivery device 102, including operational state information, condition information, and/or identity information. When the lock 141 is activated by the controller 150, the lock 141 may be configured to limit or prevent movement of the actuator 140 relative to the housing 110. When the lock 141 is deactivated by the controller 150, the lock 141 may be configured to allow movement of the actuator 140 relative to the housing 110. Once actuated by actuator 140, the controller 150 may control the drive 130 in accordance with the decision logic described with respect to FIG. 5.

The drug delivery device 102 may also include a removable sterile barrier or signal cap 144 that is disposed about one or more of a distal end of the housing 110, the needle shield 122, and the second end 118 of the delivery cannula 114. The signal cap 144 may be removably attached to the distal end of the housing 110 as shown in FIG. 1. In some embodiments, the signal cap 144 may form an interference or snap fit with the distal end of the housing 110. A frictional force associated with the interference or snap fit may be overcome by manually pulling the signal cap 144 in a direction away from a housing 110. The signal cap 144, when attached to the drug delivery device 102, may reduce the risk of contamination of the delivery cannula 114 and other elements disposed within the drug delivery device 102.

Additionally, the drug delivery device 102 may include a heating element 146 coupled to the exterior of the reservoir 112 and configured to warm the medicament inside the reservoir 112 through, for example, conductive heating. The heating element 146 may be coupled to the controller 150 so that the controller 150 can selectively activate or deactivate the heating element 146 based on different types of information regarding the drug delivery device 102, including operational state information, condition information, and/or identity information. In some embodiments, the heating element 146 may include an electrically conductive coil that is wrapped around the exterior of the reservoir 112. In other embodiments, the heating element may include an electrically conductive coil wrapped around the cannula 114. Alternatively, or additionally, a cooling element (not illustrated) may be coupled to the reservoir 112 and controllable by the controller 150 in a manner similar to the heating element 146.

The drug delivery device 102 may also include an output unit 147 coupled to the housing 110 and configured to notify the patient or user of information related to the drug delivery device 102. The output unit 147 may be coupled to the controller 150 so that the controller 150 can selectively activate or deactivate the output unit 147 based on different types of information regarding the drug delivery device 102, including operational state information, condition information, and/or identity information. The output unit 147 may be any device suitable for conveying information to the patient or user including a display (e.g., a liquid crystal display), a touchscreen, a light (e.g., a light emitting diode), a vibrator (e.g., an electro-mechanical vibrating element), a speaker, and/or an alarm, among other devices. The drug delivery device 102 may also include an input unit 148 coupled to the housing 110 and configured to allow a user or patient to input information to be used by the controller 150. In some embodiments, the input unit 148, the output unit 147, and even the fingerprint sensor 165, may be a single device such as a touchscreen. In other embodiments, the input unit 148 may be a separate device from the output unit 147 such as a keyboard or button.

The combined input unit 148 and output unit 147 (such as a single touchscreen), or the two units 147 and 148 in cooperation, may be used to implement the decision logic described with respect to FIG. 5 and to inform a user about the information calculated or received by the controller 150 as part of the decision logic described with respect to FIG. 5. For example, the output unit 147 may inform a user which drug was identified as being contained in the reservoir. The output unit 147 might display the acceptable range of injection times calculated for the identified drug and/or might display relative injection speed options to the user. The relative injection speed options may be displayed as written adjectives (e.g., slow, medium, fast), a percentage (25%, 50%, or 75% of the longest possible injection time), or a point on a sliding scale. The input unit 146 may be used to select a preferred injection speed (e.g., medium, 50% of the longest possible injection time, the middle point on the sliding scale). In some instances, the input unit 146 may have unique physical buttons associated with each relative injection speed option (such as speed selector switch 316, described below). The output unit 147 might display the preferred injection speed selected by the user or might display the tailored injection time determined by the controller. In some embodiments, the output unit 147 may display a timer counting down from the tailored injection time so that a user knows how much time remains before the injection is completed.

As illustrated in FIG. 1, the reservoir 112, the biasing element 124, the locks 128, 135, 141, the plunger 131, the stopper 132, the drive 130, and the heating element 146 are disposed within the housing 110, along with at least part of the delivery cannula 114. Also disposed within the housing 110 is a controller 150, a communication module 152 (e.g., a wireless transmitter), and at least one sensor or switch. According to the embodiment illustrated in FIG. 1, four sensors are included: a temperature sensor 160, a skin sensor 162, at least one orientation sensor 164, and a fingerprint sensor 165. The sensors 160, 162, 164, and 165 may each generate sensor data (e.g., raw or unprocessed data) related to a respective measured property or aspect of the drug delivery device 102. The sensor data may be representative of at least one of a condition or operational state of the drug delivery device 102. Additionally, the drug delivery device 102 includes a switch 166. The controller 150 is coupled to the communication module 152, the locks 128, 135, 141, the sensors 160, 162, 164, 165, the heating element 146, the fingerprint sensor 165, the output unit 147, the input unit 148, and the switch 166. The controller 150 may be configured to process the sensor data generated by the sensors 160, 162, 164, and 165 to determine a condition and/or operational state of the drug delivery device 102. The controller 150, the communication module 152, one or more of the sensors 160, 162, 164, 165 and the switch 166 may be packaged together as a single module, or each component may be fabricated separately and coupled once the components are disposed within the housing 110. According to certain embodiments, each electrical component may be integrated into the structure of the device 102 associated with that electrical component (e.g., the sensors 162 and 164 may be integrated into the shield 122). In some embodiments, the controller 150, the communication module 152, one or more of the sensors 160, 162, 164, 165, and/or the switch 166 may be packaged together inside the signal cap 144.

The controller 150 may include at least one processor 170 (e.g., a microprocessor) and a memory 172 (e.g., a random access memory (RAM), a non-volatile memory such as a hard disk, a flash memory, a removable memory, a non-removable memory, etc.). The controller 150 may also include or be coupled to a power supply, e.g. a battery. The processor 170 may be programmed to carry out the actions that the controller 150 is adapted to perform and the memory 172 may include one or more tangible non-transitory readable memories having executable, computer-readable, non-transitory instructions stored thereon, which instructions when executed by the at least one processor 170 may cause the at least one processor 170 to carry out the actions that the controller 150 is adapted to perform. Alternatively, the controller 150 may include other circuitry that carries out the actions that the controller is adapted to perform. In particular, the controller 150 may be adapted to carry out the decision logic described below with respect to FIG. 5.

The communication module 152 (i.e., reader) may be any of a number of different communication modules used to receive information from a cassette having a reservoir containing a drug to be injected (such as cassette 200, discussed below). For example, the communication module may be QR code reader, an RFID tag reader, or a near field communication (NFC) reader. The communication module 152 is used to identify the drug to be injected by reading an identifier (e.g., a QR code, RFID tag, or NFC tag) provided on the cassette having the reservoir (such as cassette 200, discussed below).

In some embodiments, the memory 172 of the controller 150 may store a drug dataset having a list of possible drugs, each possible drug associated with drug information. The drug dataset may be stored in the memory 172 prior to the start of execution of any of the methods discussed below. The drug information may include, by way of example and not by way of limitation, viscosity (at room temperature or a variety of viscosities associated with different temperatures), dose volume, minimum injection time to achieve drug efficacy, maximum injection time to achieve drug efficacy, minimum injection time to curtail injection pain, and maximum injection time to curtail injection pain. With this information, the controller 150 may calculate an acceptable range of injection times for an identified drug and may, once a preferred injection speed is received from the input unit 148, determine a tailored injection time within the acceptable range of injection times based on the preferred injection speed.

In other embodiments, the drug information discussed above may be contained in the identifier (e.g., the QR code, RFID tag, or NFC tag) provided on the cassette having the reservoir (such as cassette 200, discussed below). In such an embodiment, a full drug dataset is unnecessary. Only the drug information associated with the particular drug contained within the reservoir need be provided.

The temperature sensor 160 may be disposed proximate to the reservoir 112 so that the temperature of the drug in the reservoir 112 may be determined. Alternatively, the temperature sensor 160 may simply be disposed in the housing 110, so that an approximate temperature of the drug in the reservoir 112 and of the drug delivery device 102 generally may be determined. According to an embodiment, the temperature sensor 160 may be an on-board temperature sensor 160 attached to the processor 170.

The skin sensor 162 may be attached to or associated with the shield 122 to determine when the drug delivery device 102 is disposed on or against the patient's skin. According to one embodiment, the skin sensor 162 is a pressure sensor. According to other embodiments, the skin sensor 162 may be a capacitance sensor, resistance sensor, or inductance sensor. The skin sensor 162 or the switch 166 (which is attached to or associated with the actuator 140) may be used to determine when the drug delivery device 102 is activated or actuated, depending on the design and operation of the drug delivery device 102 that is used to actuate the drive 130, in accordance with the discussion above. It may also be the case that a signal from the skin sensor 160 is used to determine that the drug delivery device 102 has been activated even when the shield 122 is not used as the actual actuator, the underlying assumption being that the movement of the shield 122 is necessarily related to the actuation of the device 102.

The orientation sensors 164, of which there may be at least two as illustrated, may be associated with the shield 122 (or that portion of the housing 110 adjacent the shield 122) and the controller 150 (which may be, as illustrated, disposed at the other end of the drug delivery device 102 or the housing 110 from the shield 122). The orientation sensors 164 may be magnetometers, for example. In particular, the orientation sensor 164 associated with the controller 150 may be an on-board magnetometer. The orientation sensors 164 may be used to determine the orientation of the drug delivery device 102 (in particular, the housing 110) relative to the injection site (or more particularly, relative to the placement of the drug delivery device 102 on or against the patient's skin).

It will be recognized that the arrangement of the components of the drug delivery device 102 within the housing 110 is but one embodiment of this disclosure. For example, certain components of the drug delivery device 102 may be disposed outside the drug delivery device 102.

According to this embodiment, the drug delivery device 102 may include the housing 110, the reservoir 112, the needle 114, the shield 122, the biasing element 124, the lock 128, the drive 130, and the button 140. Furthermore, the sensors 162, 164 and the switch 166 may be disposed within the housing 110. The fingerprint sensor 165, the output unit 147, and the input unit 148 may be disposed on the exterior of the module 130 so that a user or patient can interact with them.

The separation of the controller 150, communication module 152 and other components into a module may permit the module to be used with multiple instances of the drug delivery device 102. In this regard, the module may be considered to be the reusable portion of the drug delivery device 102/module combination (which may be referred to as the drug delivery device 102 for purposes of this disclosure), while the drug delivery device 102 may be considered to be the disposable portion of the drug delivery device 102. By isolating the more expensive components into the reusable module 400 and the less expensive components (including certain sensors) into the disposable drug delivery device 102, the overall cost of the autoinjector may be optimized. This arrangement of the components in the module and the drug delivery device 102 may also facilitate the manufacture and sterilization of the drug delivery device 102 and module.

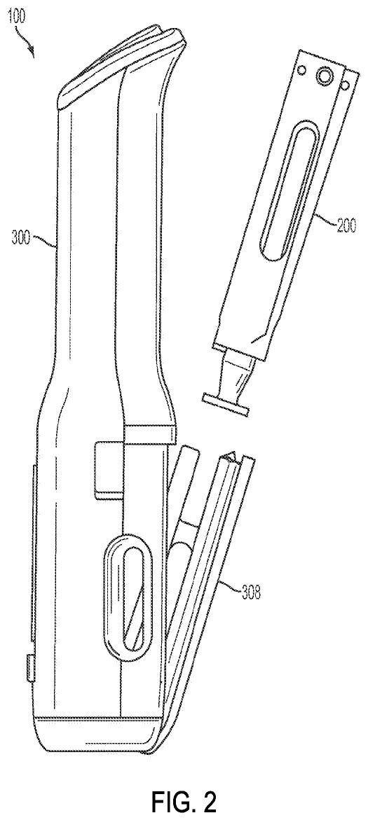

Turning to FIG. 2, an embodiment of another generalized example of a system 100′, identical or different than the generalized example of a system 100 described with respect to FIG. 1, is provided. As shown, the autoinjection system or apparatus 100′ may comprise a removable cassette 200 and an autoinjector or injector 300. Various embodiments of the cassette 200 may be constructed to contain a drug to be injected into the user by the autoinjector 300. In various other embodiments the cassette 200 may be constructed for use in training the user to operate the autoinjector 300 (a training cassette). The autoinjector 300 may be constructed to deliver an injection automatically upon actuation by the user or some other person. Various embodiments of the autoinjector 300 may have a cassette door 308 that can be constructed to pivot between and an open position and a closed position to allow insertion of the cassette 200 into the autoinjector 300. In some embodiments, the cassette door 308 may include a “cassette” icon (not shown) that indicates the insertion entry point for the cassette 200.

Referring collectively to FIGS. 3A-3G, various embodiments of the autoinjector 300 may comprise a casing 302 having a handle section 304 and a cassette receiving section 306 inline with the handle section 304. To aid patients with manual dexterity issues, the handle section 304 of the autoinjector casing 302 may define an ergonomically shaped handle 305 with a soft grip area 305S. The cassette receiving section 306 comprises the cassette door 308 described earlier. The cassette door receives the cassette 200 in an open position and aligns the cassette 200 with the drive(s), and other structures and components of the autoinjector 300 in a closed position. The cassette door 308 may include a “cassette” icon that indicates the insertion entry point for the cassette 200. The cassette receiving section 306 of the casing 302 may comprise windows 310A, 310B on sides thereof that align with windows of the cassette 200 when the cassette door 308 is closed with the cassette 200 correctly installed therein. In one or more embodiments, the windows 310A, 310B may be double-layered.

Referring still to FIGS. 3A, 3B, 3D, and 3F, the autoinjector 300 may further comprise a user interface 312 and an audio speaker (not shown). The audio speaker may be disposed inside the casing 302 and provide various audible indicators. The audio speaker may audibly communicate with the external environment via a speaker aperture 314 formed in the casing 302 in the cassette receiving section 306. The visual and audible indicators generated by the user interface 312 and the audio speaker can tell the user when the autoinjector 300 is ready for use, the progress of the injection process, injection completion, the occurrence of any errors, and other information.

The user interface 312 (best illustrated in FIG. 3A) may be located in the cassette receiving section 306 of the casing 302, and provides various visual indicators. The user interface 312 corresponds with the input unit 148 and output unit 147 described with respect to FIG. 1 and may be used to implement the same functionality. For example, the user interface 312 may inform a user which drug was identified as being contained in the reservoir. The user interface 312 might display the acceptable range of injection times calculated for the identified drug and/or might display relative injection speed options to the user. The relative injection speed options may be displayed as written adjectives (e.g., slow, medium, fast), a percentage (25%, 50%, or 75% of the longest possible injection time), or a point on a sliding scale. The user interface 312 may be used to select a preferred injection speed (e.g., medium, 50% of the longest possible injection time, the middle point on the sliding scale). (Alternately, as discussed below, a speed selector switch 316 may be used to select a preferred injection speed.) The user interface 312 might display the preferred injection speed selected by the user or might display the tailored injection time determined by the controller. In some embodiments, the user interface 312 may display a timer counting down from the tailored injection time so that a user knows how much time remains before the injection is completed.

The autoinjector 300 may further comprise one or more of a settings/mute switch 315, a speed selector switch 316, a start button 307, and an eject button 317. The settings/mute switch 315 (FIG. 3B) may be located in the cassette receiving section 306 of the casing 302. The mute switch 315 may be constructed allow the user to turn on and off all synthesized sounds, except error sounds, and to respond in real-time so that if the user begins the injection process and changes the mute switch to off, the sounds are immediately muted. The mute switch 315 may also be constructed to slide toward a “mute” icon to mute the audio speaker. A light indicator may be provided to confirm the “mute” state.

The speed selector switch 316 (FIGS. 3A and 3B) may be located in the cassette receiving section 306 of the casing 302. The speed selector switch 316 may be constructed to allow the user to select among a plurality of relative injection speed options. The relative injection speed option selected by the user can be used in the decision logic described in FIG. 5 as the preferred injection speed. The speed selector switch 316 may comprise a three switch positions. Other embodiments of the speed selector switch may comprise two switch positions, or 4 or more switch positions. In still other embodiments, the speed selector switch may be of the infinitely variable type. The autoinjector 300 may also be provided with one or more demo cassettes to allow the user to experiment with selecting different relative injection speed options as the preferred injection speed

The start button 307 may be disposed at a free end of the handle 305. The button 307 may be made of a translucent material that allows a lighting effect to illuminate the button as signals. The eject button 317 (FIG. 3D) may be located in the cassette receiving section 306 of the casing 302. In some embodiments, the eject button 317 may be controlled by the microprocessor 350 (FIG. 3G) of the autoinjector 300, which may be programmed to eliminate accidental inputs during the injection process.

Referring to FIG. 3E, the cassette receiving section 306 of the casing 302 and the cassette door 308 may form a proximal end wall 318 of the autoinjector 300. The proximal end wall 318 may be configured as a broad, flat and stable base for easily positioning the autoinjector 300 on a support surface, after removal of the shield remover 240 or when the autoinjector 300 does not contain the cassette 240. The portion of the proximal end wall 318 formed by the cassette door 308 may include an aperture 308A that is sized and shaped to allow the shield remover 240 to be removed from the cassette 200 and withdrawn through the aperture 308A, when the cassette 200 is installed in the autoinjector 300. The proximal end wall of the autoinjector 300 may further comprise a target light 320.

As shown in FIG. 3G, various embodiments of the autoinjector 300 may comprise a chassis 301 disposed in the casing 302 for supporting a motorized needle insertion drive 330, a motorized drive 340, a microprocessor 350, a battery 360 for powering the drives 330, 340 and the microprocessor 350, and the skin sensor 380. The casing 302 may define an ergonomically shaped handle section 304 and a cassette receiving section 306. The chassis 301 may include a support surface 301s for supporting one or more cassettes 200 in the autoinjector 300 and aligning the cassette 200 or a selected one of the one or more cassettes 200 with motorized drives 330 and 340, respectively.

A detector 370 (i.e., reader) may be provided on or in the cassette support surface 301s for sensing the presence of and/or information about the cassette 200. The detector 370 corresponds with the communication module 152 described with respect to FIG. 1 and may be used to implement the same functionality. In particular, the detector 370 may be used to read an identifier 211 of the cassette 200, discussed in more detail below. The detector 370 may be coupled with the microprocessor 350 (i.e., controller 350) in a manner that allows signals or data to be communicated to the microprocessor 350. The microprocessor 350 corresponds with the controller 150 described with respect to FIG. 1 and may be used to implement the same functionality.

The insertion drive 330 may include an insertion rack 332, an insertion drive motor 331 and an insertion drive gear train 333 for transmitting rotary motion of the insertion drive motor 331 to drive the rack 332. The insertion rack may include a tab arrangement including, for example, proximal and distal tabs 332p and 332d, respectively, which interface with the cassette 200. The drive 340 may comprise a drive motor 341, a plunger rod 342, a lead screw 343, and a drive gear train 344. The plunger rod 342 is driven by the drive motor 341 through the lead screw 343 and the drive gear train 344, and may interface with a plunger 264 of a drug container 260 contained within the cassette 200. The autoinjector 300 can be used for executing multiple injections.

Referring still to FIG. 3G, the microprocessor 350 of the autoinjector 300 may be programmed with instructions that, when executed by the microprocessor 350, enable it to control and monitor the various operations and functions of the autoinjector 300. For example, but not limitation, the microprocessor 350 may be programmed with instructions for controlling the drives 330, 340. Specifically, the microprocessor 350 may be programmed with instructions for implementing the decision logic described below with respect to FIG. 5.

In various other embodiments, the autoinjector 300 may include other types of drives and means for activating and sequencing the drives. The drives in such embodiments may be implemented as separate and distinct mechanisms or combined into a single mechanism. The drives of such embodiments may be powered, without limitation, by motors, mechanical mechanisms (e.g., elastic members such as springs), gas pressure mechanisms, gas releasing mechanism, or any combination thereof. Various transmission mechanisms may be used for transmitting the power to the cassette, to cause injection of the drug. In addition, the activating and sequencing means may comprise various mechanical and electromechanical arrangements, which may be combined with the microprocessor described earlier or used alone. The autoinjector in such embodiments may be constructed to be reusable for executing multiple injections or be designed for a single, disposable use.

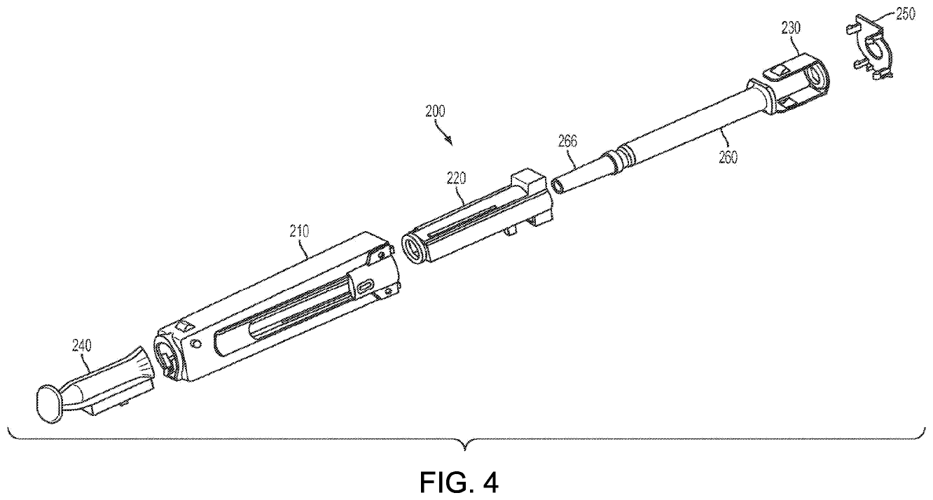

Referring now to FIG. 4, various embodiments of the cassette 200 may comprise an outer housing 210, an inner sleeve 220, a drug container 260 for containing a drug, a cassette cap 240, a lock cap 230, and a cover 250. Such embodiments of the cassette 200 facilitate and enable easy injection of the drug with the autoinjector and can be constructed for a single, disposable use. In various embodiments, the lock cap 230 and cover 250 of the cassette 200 may be constructed to resist removal of the drug container 260 from the cassette 200, thereby preventing needle sticks before and after use of the cassette 200 and also preventing the drug container 260 from being taken out of the cassette 200 or replaced. In addition, the lock cap 230 and cover 250 protect the drug container 260 during shipment and transportation. The cassette cap 240, in various embodiments, may be constructed to remove a needle shield 266 covering an injection needle associated with the drug container 260. In various other embodiments, the cassette cap 240 may also be constructed to engage the outer housing 210 of the cassette 200, such that the cassette cap 240 cannot be rotated or twisted, thereby preventing the needle shield 266 from damaging the injection needle. Various embodiments of the inner sleeve 220 may be constructed to position the drug container 260 within the cassette housing 210 in either a needle-concealed position or a needle injection position during an injection cycle of the autoinjector. In various other embodiments, the outer housing 210 and the inner sleeve 220 of the cassette 200 may include one or more locking arrangements that protect the drug container 260 and prevent unintended needle exposure or damage.

The cassette 200 may include an identifier 211 on outer housing 210. The identifier 211 may be placed such that the identifier 211 can be read by detector 370 when the cassette 200 is placed in the autoinjector 300. The identifier 211 may be, for example, a QR code, an RFID tag, or an NFC tag. The identifier 211 specifies the drug contained within the cassette 200. The identifier 211 may further include drug information associated with the particular drug contained within the cassette 200. For example, the identifier may provide drug information including but not limited to viscosity (at room temperature or a variety of viscosities associated with different temperatures), dose volume, minimum injection time to achieve drug efficacy, maximum injection time to achieve drug efficacy, minimum injection time to curtail injection pain, and maximum injection time to curtail injection pain.

FIG. 5 illustrates a method 500 for injecting a drug using the autoinjector (such as autoinjector 300 or drug delivery device 102) over a tailored injection time. At box 502, the method 500 includes identifying a drug contained in a reservoir of a drug delivery system, which may be based on a reading of drug information by a reader of the drug delivery system. At box 504, the method 500 includes calculating or determining an acceptable range of injection times for the identified drug. At box 506, the method 500 includes receiving a preferred injection speed from an input device of the drug delivery system. At box 508, the method 500 includes determining a tailor injection time within the acceptable range of injection times, which may be based on the preferred injection speed. At box 510, the method 500 includes setting a delivery speed of a drive of the drug delivery system such that the drug is expelled from the reservoir over the tailored injection time.

At box 504, calculating or determining the acceptable range of injection times for the identified drug may include receiving drug information from the identifier on the reservoir. Alternately, or in addition, at box 504, calculating or determining the acceptable range of injection times for the identified drug may include accessing a drug information dataset provided in a memory of the controller, determining that the identified drug is one possible drug in a list of possible drugs, and receiving drug information associated with the identified drug in the drug information dataset. Either way, the drug information may include at least one of viscosity, dose volume, minimum injection time to achieve drug efficacy, maximum injection time to achieve drug efficacy, minimum injection time to curtail injection pain, and maximum injection time to curtail injection pain.

The method 500 may further include presenting two or more relative injection speed options, each relative injection speed option equal to a percentage of a longest possible injection time within the range of acceptable injection times, allowing selection of one of the two or more relative injection speed options, and using the relative injection speed option selected as the preferred injection speed. Each of the two or more relative injection speed options may be displayed as one of a written adjective, the percentage of the longest possible injection, and a point on a sliding scale. The method 500 may further include causing the input device to display the two or more relative injection speed options on a touchscreen. Alternately or in addition, selection of one of the two or more relative injection speed options may include associating the two or more relative injection speeds with unique physical buttons of the drug delivery device. Identifying the drug contained in the reservoir of the drug delivery system based on the reading by the reader of the drug delivery system may include reading an RFID tag or an NFC tag.

It will be appreciated that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. The same reference numbers may be used to describe like or similar parts. Further, while several examples have been disclosed herein, any features from any examples may be combined with or replaced by other features from other examples. Moreover, while several examples have been disclosed herein, changes may be made to the disclosed examples within departing from the scope of the claims.

The above description describes various drug delivery devices and methods for use with a drug delivery device. It should be clear that the drug delivery devices or methods can further comprise use of a medicament listed below with the caveat that the following list should neither be considered to be all inclusive nor limiting. The medicament will be contained in a reservoir. In some instances, the reservoir is a primary container that is either filled or pre-filled for treatment with the medicament. The primary container can be a cartridge or a pre-filled syringe.

For example, the drug delivery device or more specifically the reservoir of the device may be filled with colony stimulating factors, such as granulocyte colony-stimulating factor (G-CSF). Such G-CSF agents include, but are not limited to, Neupogen® (filgrastim) and Neulasta® (pegfilgrastim). In various other embodiments, the drug delivery device may be used with various pharmaceutical products, such as an erythropoiesis stimulating agent (ESA), which may be in a liquid or a lyophilized form. An ESA is any molecule that stimulates erythropoiesis, such as Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin zeta, epoetin theta, and epoetin delta, as well as the molecules or variants or analogs thereof as disclosed in the following patents or patent applications, each of which is herein incorporated by reference in its entirety: U.S. Pat. Nos. 4,703,008; 5,441,868; 5,547,933; 5,618,698; 5,621,080; 5,756,349; 5,767,078; 5,773,569; 5,955,422; 5,986,047; 6,583,272; 7,084,245; and 7,271,689; and PCT Publication Nos. WO 91/05867; WO 95/05465; WO 96/40772; WO 00/24893; WO 01/81405; and WO 2007/136752.

An ESA can be an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, epoetin alfa, epoetin beta, epoetin delta, epoetin omega, epoetin iota, epoetin zeta, and analogs thereof, pegylated erythropoietin, carbamylated erythropoietin, mimetic peptides (including EMP1/hematide), and mimetic antibodies. Exemplary erythropoiesis stimulating proteins include erythropoietin, darbepoetin, erythropoietin agonist variants, and peptides or antibodies that bind and activate erythropoietin receptor (and include compounds reported in U.S. Publication Nos. 2003/0215444 and 2006/0040858, the disclosures of each of which is incorporated herein by reference in its entirety) as well as erythropoietin molecules or variants or analogs thereof as disclosed in the following patents or patent applications, which are each herein incorporated by reference in its entirety: U.S. Pat. Nos. 4,703,008; 5,441,868; 5,547,933; 5,618,698; 5,621,080; 5,756,349; 5,767,078; 5,773,569; 5,955,422; 5,830,851; 5,856,298; 5,986,047; 6,030,086; 6,310,078; 6,391,633; 6,583,272; 6,586,398; 6,900,292; 6,750,369; 7,030,226; 7,084,245; and 7,217,689; U.S. Publication Nos. 2002/0155998; 2003/0077753; 2003/0082749; 2003/0143202; 2004/0009902; 2004/0071694; 2004/0091961; 2004/0143857; 2004/0157293; 2004/0175379; 2004/0175824; 2004/0229318; 2004/0248815; 2004/0266690; 2005/0019914; 2005/0026834; 2005/0096461; 2005/0107297; 2005/0107591; 2005/0124045; 2005/0124564; 2005/0137329; 2005/0142642; 2005/0143292; 2005/0153879; 2005/0158822; 2005/0158832; 2005/0170457; 2005/0181359; 2005/0181482; 2005/0192211; 2005/0202538; 2005/0227289; 2005/0244409; 2006/0088906; and 2006/0111279; and PCT Publication Nos. WO 91/05867; WO 95/05465; WO 99/66054; WO 00/24893; WO 01/81405; WO 00/61637; WO 01/36489; WO 02/014356; WO 02/19963; WO 02/20034; WO 02/49673; WO 02/085940; WO 03/029291; WO 2003/055526; WO 2003/084477; WO 2003/094858; WO 2004/002417; WO 2004/002424; WO 2004/009627; WO 2004/024761; WO 2004/033651; WO 2004/035603; WO 2004/043382; WO 2004/101600; WO 2004/101606; WO 2004/101611; WO 2004/106373; WO 2004/018667; WO 2005/001025; WO 2005/001136; WO 2005/021579; WO 2005/025606; WO 2005/032460; WO 2005/051327; WO 2005/063808; WO 2005/063809; WO 2005/070451; WO 2005/081687; WO 2005/084711; WO 2005/103076; WO 2005/100403; WO 2005/092369; WO 2006/50959; WO 2006/02646; and WO 2006/29094.

Examples of other pharmaceutical products for use with the device may include, but are not limited to, antibodies such as Vectibix® (panitumumab), Xgeva™ (denosumab) and Prolia™ (denosamab); other biological agents such as Enbrel® (etanercept, TNF-receptor/Fc fusion protein, TNF blocker), Neulasta® (pegfilgrastim, pegylated filgastrim, pegylated G-CSF, pegylated hu-Met-G-CSF), Neupogen® (filgrastim, G-CSF, hu-MetG-CSF), and Nplate® (romiplostim); small molecule drugs such as Sensipar® (cinacalcet). The device may also be used with a therapeutic antibody, a polypeptide, a protein or other chemical, such as an iron, for example, ferumoxytol, iron dextrans, ferric glyconate, and iron sucrose. The pharmaceutical product may be in liquid form, or reconstituted from lyophilized form.

Among particular illustrative proteins are the specific proteins set forth below, including fusions, fragments, analogs, variants or derivatives thereof:

OPGL specific antibodies, peptibodies, and related proteins, and the like (also referred to as RANKL specific antibodies, peptibodies and the like), including fully humanized and human OPGL specific antibodies, particularly fully humanized monoclonal antibodies, including but not limited to the antibodies described in PCT Publication No. WO 03/002713, which is incorporated herein in its entirety as to OPGL specific antibodies and antibody related proteins, particularly those having the sequences set forth therein, particularly, but not limited to, those denoted therein: 9H7; 18B2; 2D8; 2E11; 16E1; and 22B3, including the OPGL specific antibodies having either the light chain of SEQ ID NO:2 as set forth therein in FIG. 2 and/or the heavy chain of SEQ ID NO:4, as set forth therein in FIG. 4, each of which is individually and specifically incorporated by reference herein in its entirety fully as disclosed in the foregoing publication;

Myostatin binding proteins, peptibodies, and related proteins, and the like, including myostatin specific peptibodies, particularly those described in U.S. Publication No. 2004/0181033 and PCT Publication No. WO 2004/058988, which are incorporated by reference herein in their entirety particularly in parts pertinent to myostatin specific peptibodies, including but not limited to peptibodies of the mTN8-19 family, including those of SEQ ID NOS:305-351, including TN8-19-1 through TN8-19-40, TN8-19 con1 and TN8-19 con2; peptibodies of the mL2 family of SEQ ID NOS:357-383; the mL15 family of SEQ ID NOS:384-409; the mL17 family of SEQ ID NOS:410-438; the mL20 family of SEQ ID NOS:439-446; the mL21 family of SEQ ID NOS:447-452; the mL24 family of SEQ ID NOS:453-454; and those of SEQ ID NOS:615-631, each of which is individually and specifically incorporated by reference herein in their entirety fully as disclosed in the foregoing publication;

IL-4 receptor specific antibodies, peptibodies, and related proteins, and the like, particularly those that inhibit activities mediated by binding of IL-4 and/or IL-13 to the receptor, including those described in PCT Publication No. WO 2005/047331 or PCT Application No. PCT/US2004/37242 and in U.S. Publication No. 2005/112694, which are incorporated herein by reference in their entirety particularly in parts pertinent to IL-4 receptor specific antibodies, particularly such antibodies as are described therein, particularly, and without limitation, those designated therein: L1H1; L1H2; L1H3; L1H4; L1H5; L1H6; L1H7; L1H8; L1H9; L1H10; L1H11; L2H1; L2H2; L2H3; L2H4; L2H5; L2H6; L2H7; L2H8; L2H9; L2H10; L2H11; L2H12; L2H13; L2H14; L3H1; L4H1; L5H1; L6H1, each of which is individually and specifically incorporated by reference herein in its entirety fully as disclosed in the foregoing publication;

Interleukin 1-receptor 1 (“IL1-R1”) specific antibodies, peptibodies, and related proteins, and the like, including but not limited to those described in U.S. Publication No. 2004/097712, which is incorporated herein by reference in its entirety in parts pertinent to IL1-R1 specific binding proteins, monoclonal antibodies in particular, especially, without limitation, those designated therein: 15CA, 26F5, 27F2, 24E12, and 10H7, each of which is individually and specifically incorporated by reference herein in its entirety fully as disclosed in the aforementioned publication;

Ang2 specific antibodies, peptibodies, and related proteins, and the like, including but not limited to those described in PCT Publication No. WO 03/057134 and U.S. Publication No. 2003/0229023, each of which is incorporated herein by reference in its entirety particularly in parts pertinent to Ang2 specific antibodies and peptibodies and the like, especially those of sequences described therein and including but not limited to: L1(N); L1(N) WT; L1(N) 1K WT; 2xL1(N); 2xL1(N) WT; Con4 (N), Con4 (N) 1K WT, 2xCon4 (N) 1K; L1C; L1C 1K; 2xL1C; Con4C; Con4C 1K; 2xCon4C 1K; Con4-L1 (N); Con4-L1C; TN-12-9 (N); C17 (N); TN8-8(N); TN8-14 (N); Con 1 (N), also including anti-Ang 2 antibodies and formulations such as those described in PCT Publication No. WO 2003/030833 which is incorporated herein by reference in its entirety as to the same, particularly Ab526; Ab528; Ab531; Ab533; Ab535; Ab536; Ab537; Ab540; Ab543; Ab544; Ab545; Ab546; A551; Ab553; Ab555; Ab558; Ab559; Ab565; AbF1AbFD; AbFE; AbFJ; AbFK; AbG1D4; AbGC1E8; AbH1C12; AbIA1; AbIF; AbIK, AbIP; and AbIP, in their various permutations as described therein, each of which is individually and specifically incorporated by reference herein in its entirety fully as disclosed in the foregoing publication;

NGF specific antibodies, peptibodies, and related proteins, and the like including, in particular, but not limited to those described in U.S. Publication No. 2005/0074821 and U.S. Pat. No. 6,919,426, which are incorporated herein by reference in their entirety particularly as to NGF-specific antibodies and related proteins in this regard, including in particular, but not limited to, the NGF-specific antibodies therein designated 4D4, 4G6, 6H9, 7H2, 14D10 and 14D11, each of which is individually and specifically incorporated by reference herein in its entirety fully as disclosed in the foregoing publication;

CD22 specific antibodies, peptibodies, and related proteins, and the like, such as those described in U.S. Pat. No. 5,789,554, which is incorporated herein by reference in its entirety as to CD22 specific antibodies and related proteins, particularly human CD22 specific antibodies, such as but not limited to humanized and fully human antibodies, including but not limited to humanized and fully human monoclonal antibodies, particularly including but not limited to human CD22 specific IgG antibodies, such as, for instance, a dimer of a human-mouse monoclonal hLL2 gamma-chain disulfide linked to a human-mouse monoclonal hLL2 kappa-chain, including, but limited to, for example, the human CD22 specific fully humanized antibody in Epratuzumab, CAS registry number 501423-23-0;

IGF-1 receptor specific antibodies, peptibodies, and related proteins, and the like, such as those described in PCT Publication No. WO 06/069202, which is incorporated herein by reference in its entirety as to IGF-1 receptor specific antibodies and related proteins, including but not limited to the IGF-1 specific antibodies therein designated L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, L52H52, and IGF-1R-binding fragments and derivatives thereof, each of which is individually and specifically incorporated by reference herein in its entirety fully as disclosed in the foregoing publication;

Also among non-limiting examples of anti-IGF-1R antibodies for use in the methods and compositions of the present invention are each and all of those described in:

U.S. Publication No. 2006/0040358 (published Feb. 23, 2006), 2005/0008642 (published Jan. 13, 2005), 2004/0228859 (published Nov. 18, 2004), including but not limited to, for instance, antibody 1A (DSMZ Deposit No. DSM ACC 2586), antibody 8 (DSMZ Deposit No. DSM ACC 2589), antibody 23 (DSMZ Deposit No. DSM ACC 2588) and antibody 18 as described therein;

PCT Publication No. WO 06/138729 (published Dec. 28, 2006) and WO 05/016970 (published Feb. 24, 2005), and Lu et al. (2004), J. Biol. Chem. 279:2856-2865, including but not limited to antibodies 2F8, A12, and IMC-A12 as described therein;

PCT Publication No. WO 07/012614 (published Feb. 1, 2007), WO 07/000328 (published Jan. 4, 2007), WO 06/013472 (published Feb. 9, 2006), WO 05/058967 (published Jun. 30, 2005), and WO 03/059951 (published Jul. 24, 2003);

U.S. Publication No. 2005/0084906 (published Apr. 21, 2005), including but not limited to antibody 7C10, chimaeric antibody C7C10, antibody h7C10, antibody 7H2M, chimaeric antibody *7C10, antibody GM 607, humanized antibody 7C10 version 1, humanized antibody 7C10 version 2, humanized antibody 7C10 version 3, and antibody 7H2HM, as described therein;

U.S. Publication Nos. 2005/0249728 (published Nov. 10, 2005), 2005/0186203 (published Aug. 25, 2005), 2004/0265307 (published Dec. 30, 2004), and 2003/0235582 (published Dec. 25, 2003) and Maloney et al. (2003), Cancer Res. 63:5073-5083, including but not limited to antibody EM164, resurfaced EM164, humanized EM164, huEM164 v1.0, huEM164 v1.1, huEM164 v1.2, and huEM164 v1.3 as described therein;

U.S. Pat. No. 7,037,498 (issued May 2, 2006), U.S. Publication Nos. 2005/0244408 (published Nov. 30, 2005) and 2004/0086503 (published May 6, 2004), and Cohen, et al. (2005), Clinical Cancer Res. 11:2063-2073, e.g., antibody CP-751,871, including but not limited to each of the antibodies produced by the hybridomas having the ATCC accession numbers PTA-2792, PTA-2788, PTA-2790, PTA-2791, PTA-2789, PTA-2793, and antibodies 2.12.1, 2.13.2, 2.14.3, 3.1.1, 4.9.2, and 4.17.3, as described therein;

U.S. Publication Nos. 2005/0136063 (published Jun. 23, 2005) and 2004/0018191 (published Jan. 29, 2004), including but not limited to antibody 19D12 and an antibody comprising a heavy chain encoded by a polynucleotide in plasmid 15H12/19D12 HCA (γ4), deposited at the ATCC under number PTA-5214, and a light chain encoded by a polynucleotide in plasmid 15H12/19D12 LCF (κ), deposited at the ATCC under number PTA-5220, as described therein; and

U.S. Publication No. 2004/0202655 (published Oct. 14, 2004), including but not limited to antibodies PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT- 11A5, PINT-11A7, PINT-11A12, PINT-12A1, PINT-12A2, PINT-12A3, PINT-12A4, and PINT-12A5, as described therein; each and all of which are herein incorporated by reference in their entireties, particularly as to the aforementioned antibodies, peptibodies, and related proteins and the like that target IGF-1 receptors;

B-7 related protein 1 specific antibodies, peptibodies, related proteins and the like (“B7RP-1,” also is referred to in the literature as B7H2, ICOSL, B7h, and CD275), particularly B7RP-specific fully human monoclonal IgG2 antibodies, particularly fully human IgG2 monoclonal antibody that binds an epitope in the first immunoglobulin-like domain of B7RP-1, especially those that inhibit the interaction of B7RP-1 with its natural receptor, ICOS, on activated T cells in particular, especially, in all of the foregoing regards, those disclosed in U.S. Publication No. 2008/0166352 and PCT Publication No. WO 07/011941, which are incorporated herein by reference in their entireties as to such antibodies and related proteins, including but not limited to antibodies designated therein as follow: 16H (having light chain variable and heavy chain variable sequences SEQ ID NO:1 and SEQ ID NO:7 respectively therein); 5D (having light chain variable and heavy chain variable sequences SEQ ID NO:2 and SEQ ID NO:9 respectively therein); 2H (having light chain variable and heavy chain variable sequences SEQ ID NO:3 and SEQ ID NO:10 respectively therein); 43H (having light chain variable and heavy chain variable sequences SEQ ID NO:6 and SEQ ID NO:14 respectively therein); 41H (having light chain variable and heavy chain variable sequences SEQ ID NO:5 and SEQ ID NO:13 respectively therein); and 15H (having light chain variable and heavy chain variable sequences SEQ ID NO:4 and SEQ ID NO:12 respectively therein), each of which is individually and specifically incorporated by reference herein in its entirety fully as disclosed in the foregoing publication;

IL-15 specific antibodies, peptibodies, and related proteins, and the like, such as, in particular, humanized monoclonal antibodies, particularly antibodies such as those disclosed in U.S. Publication Nos. 2003/0138421; 2003/023586; and 2004/0071702; and U.S. Pat. No. 7,153,507, each of which is incorporated herein by reference in its entirety as to IL-15 specific antibodies and related proteins, including peptibodies, including particularly, for instance, but not limited to, HuMax IL-15 antibodies and related proteins, such as, for instance, 146B7;

IFN gamma specific antibodies, peptibodies, and related proteins and the like, especially human IFN gamma specific antibodies, particularly fully human anti-IFN gamma antibodies, such as, for instance, those described in U.S. Publication No. 2005/0004353, which is incorporated herein by reference in its entirety as to IFN gamma specific antibodies, particularly, for example, the antibodies therein designated 1118; 1118*; 1119; 1121; and 1121*. The entire sequences of the heavy and light chains of each of these antibodies, as well as the sequences of their heavy and light chain variable regions and complementarity determining regions, are each individually and specifically incorporated by reference herein in its entirety fully as disclosed in the foregoing publication and in Thakur et al. (1999), Mol. Immunol. 36:1107-1115. In addition, description of the properties of these antibodies provided in the foregoing publication is also incorporated by reference herein in its entirety. Specific antibodies include those having the heavy chain of SEQ ID NO:17 and the light chain of SEQ ID NO:18; those having the heavy chain variable region of SEQ ID NO:6 and the light chain variable region of SEQ ID NO:8; those having the heavy chain of SEQ ID NO:19 and the light chain of SEQ ID NO:20; those having the heavy chain variable region of SEQ ID NO:10 and the light chain variable region of SEQ ID NO:12; those having the heavy chain of SEQ ID NO:32 and the light chain of SEQ ID NO:20; those having the heavy chain variable region of SEQ ID NO:30 and the light chain variable region of SEQ ID NO:12; those having the heavy chain sequence of SEQ ID NO:21 and the light chain sequence of SEQ ID NO:22; those having the heavy chain variable region of SEQ ID NO:14 and the light chain variable region of SEQ ID NO:16; those having the heavy chain of SEQ ID NO:21 and the light chain of SEQ ID NO:33; and those having the heavy chain variable region of SEQ ID NO:14 and the light chain variable region of SEQ ID NO:31, as disclosed in the foregoing publication. A specific antibody contemplated is antibody 1119 as disclosed in the foregoing U.S. publication and having a complete heavy chain of SEQ ID NO:17 as disclosed therein and having a complete light chain of SEQ ID NO:18 as disclosed therein;

TALL-1 specific antibodies, peptibodies, and the related proteins, and the like, and other TALL specific binding proteins, such as those described in U.S. Publication Nos. 2003/0195156 and 2006/0135431, each of which is incorporated herein by reference in its entirety as to TALL-1 binding proteins, particularly the molecules of Tables 4 and 5B, each of which is individually and specifically incorporated by reference herein in its entirety fully as disclosed in the foregoing publications;

Parathyroid hormone (“PTH”) specific antibodies, peptibodies, and related proteins, and the like, such as those described in U.S. Pat. No. 6,756,480, which is incorporated herein by reference in its entirety, particularly in parts pertinent to proteins that bind PTH;

Thrombopoietin receptor (“TPO-R”) specific antibodies, peptibodies, and related proteins, and the like, such as those described in U.S. Pat. No. 6,835,809, which is herein incorporated by reference in its entirety, particularly in parts pertinent to proteins that bind TPO-R;

Hepatocyte growth factor (“HGF”) specific antibodies, peptibodies, and related proteins, and the like, including those that target the HGF/SF:cMet axis (HGF/SF:c-Met), such as the fully human monoclonal antibodies that neutralize hepatocyte growth factor/scatter (HGF/SF) described in U.S. Publication No. 2005/0118643 and PCT Publication No. WO 2005/017107, huL2G7 described in U.S. Pat. No. 7,220,410 and OA-5d5 described in U.S. Pat. Nos. 5,686,292 and 6,468,529 and in PCT Publication No. WO 96/38557, each of which is incorporated herein by reference in its entirety, particularly in parts pertinent to proteins that bind HGF;

TRAIL-R2 specific antibodies, peptibodies, related proteins and the like, such as those described in U.S. Pat. No. 7,521,048, which is herein incorporated by reference in its entirety, particularly in parts pertinent to proteins that bind TRAIL-R2;

Activin A specific antibodies, peptibodies, related proteins, and the like, including but not limited to those described in U.S. Publication No. 2009/0234106, which is herein incorporated by reference in its entirety, particularly in parts pertinent to proteins that bind Activin A;

TGF-beta specific antibodies, peptibodies, related proteins, and the like, including but not limited to those described in U.S. Pat. No. 6,803,453 and U.S. Publication No. 2007/0110747, each of which is herein incorporated by reference in its entirety, particularly in parts pertinent to proteins that bind TGF-beta;

Amyloid-beta protein specific antibodies, peptibodies, related proteins, and the like, including but not limited to those described in PCT Publication No. WO 2006/081171, which is herein incorporated by reference in its entirety, particularly in parts pertinent to proteins that bind amyloid-beta proteins. One antibody contemplated is an antibody having a heavy chain variable region comprising SEQ ID NO:8 and a light chain variable region having SEQ ID NO:6 as disclosed in the foregoing publication;

c-Kit specific antibodies, peptibodies, related proteins, and the like, including but not limited to those described in U.S. Publication No. 2007/0253951, which is incorporated herein by reference in its entirety, particularly in parts pertinent to proteins that bind c-Kit and/or other stem cell factor receptors;

OX40L specific antibodies, peptibodies, related proteins, and the like, including but not limited to those described in U.S. Publication No. 2006/0002929, which is incorporated herein by reference in its entirety, particularly in parts pertinent to proteins that bind OX40L and/or other ligands of the OX40 receptor; and

Other exemplary proteins, including Activase® (alteplase, tPA); Aranesp® (darbepoetin alfa); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Humatrope® (somatropin, Human Growth Hormone); Humira® (adalimumab); insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Neulasta® (pegylated filgastrim, pegylated G-CSF, pegylated hu-Met-G-CSF); Neupogen® (filgrastim, G-CSF, hu-MetG-CSF); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/IIia receptor monoclonal antibody); Actemra® (anti-IL6 Receptor mAb); Avastin® (bevacizumab), HuMax-CD4 (zanolimumab); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 146B7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Vectibix® (panitumumab); Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Rα mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-1103); anti-IGF1R mAb; anti-IGF-1R mAb (HuMax-Inflam); anti-IL12 mAb (ABT-874); anti-IL12/1L23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); anti-LLY antibody; BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; anti-ZP3 mAb (HuMax-ZP3); NVS Antibody #1; and NVS Antibody #2.

Also included can be a sclerostin antibody, such as but not limited to romosozumab, blosozumab, or BPS 804 (Novartis). Further included can be therapeutics such as rilotumumab, bixalomer, trebananib, ganitumab, conatumumab, motesanib diphosphate, brodalumab, vidupiprant, panitumumab, denosumab, NPLATE, PROLIA, VECTIBIX or XGEVA. Additionally, included in the device can be a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab), as well as molecules, variants, analogs or derivatives thereof as disclosed in the following patents or patent applications, each of which is herein incorporated by reference in its entirety for all purposes: U.S. Pat. No. 8,030,547, U.S. Publication No. 2013/0064825, WO2008/057457, WO2008/057458, WO2008/057459, WO2008/063382, WO2008/133647, WO2009/100297, WO2009/100318, WO2011/037791, WO2011/053759, WO2011/053783, WO2008/125623, WO2011/072263, WO2009/055783, WO2012/0544438, WO2010/029513, WO2011/111007, WO2010/077854, WO2012/088313, WO2012/101251, WO2012/101252, WO2012/101253, WO2012/109530, and WO2001/031007.

Also included can be talimogene laherparepvec or another oncolytic HSV for the treatment of melanoma or other cancers. Examples of oncolytic HSV include, but are not limited to talimogene laherparepvec (U.S. Pat. Nos. 7,223,593 and 7,537,924); OncoVEXGALV/CD (U.S. Pat. No. 7,981,669); OrienX010 (Lei et al. (2013), World J. Gastroenterol., 19:5138-5143); G207, 1716; NV1020; NV12023; NV1034 and NV1042 (Vargehes et al. (2002), Cancer Gene Ther., 9(12):967-978).

Also included are TIMPs. TIMPs are endogenous tissue inhibitors of metalloproteinases (TIMPs) and are important in many natural processes. TIMP-3 is expressed by various cells or and is present in the extracellular matrix; it inhibits all the major cartilage-degrading metalloproteases, and may play a role in role in many degradative diseases of connective tissue, including rheumatoid arthritis and osteoarthritis, as well as in cancer and cardiovascular conditions. The amino acid sequence of TIMP-3, and the nucleic acid sequence of a DNA that encodes TIMP-3, are disclosed in U.S. Pat. No. 6,562,596, issued May 13, 2003, the disclosure of which is incorporated by reference herein. Description of TIMP mutations can be found in U.S. Publication No. 2014/0274874 and PCT Publication No. WO 2014/152012.

Also included are antagonistic antibodies for human calcitonin gene-related peptide (CGRP) receptor and bispecific antibody molecule that target the CGRP receptor and other headache targets. Further information concerning these molecules can be found in PCT Application No. WO 2010/075238.

Additionally, bispecific T cell engager (BiTE®) antibodies, e.g. BLINCYTO® (blinatumomab), can be used in the device. Alternatively, included can be an APJ large molecule agonist e.g., apelin or analogues thereof in the device. Information relating to such molecules can be found in PCT Publication No. WO 2014/099984.

In certain embodiments, the medicament comprises a therapeutically effective amount of an anti-thymic stromal lymphopoietin (TSLP) or TSLP receptor antibody. Examples of anti-TSLP antibodies that may be used in such embodiments include, but are not limited to, those described in U.S. Pat. Nos. 7,982,016, and 8,232,372, and U.S. Publication No. 2009/0186022. Examples of anti-TSLP receptor antibodies include, but are not limited to, those described in U.S. Pat. No. 8,101,182. In particularly preferred embodiments, the medicament comprises a therapeutically effective amount of the anti-TSLP antibody designated as A5 within U.S. Pat. No. 7,982,016.

Although the drug delivery devices, methods, and elements thereof, have been described in terms of exemplary embodiments, they are not limited thereto. The detailed description is to be construed as exemplary only and does not describe every possible embodiment of the invention because describing every possible embodiment would be impractical, if not impossible. Numerous alternative embodiments could be implemented, using either current technology or technology developed after the filing date of this patent that would still fall within the scope of the claims defining the invention. For example, components described herein with reference to certain kinds of drug delivery devices, such as on-body injector drug delivery devices or other kinds of drug delivery devices, can also be utilized in other kinds of drug delivery devices, such as autoinjector drug delivery devices.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

1. A drug delivery system comprising:
a reservoir adapted to contain a drug;
an identifier having drug information;
a drug delivery device adapted to receive the reservoir;
a reader adapted to read the drug information;
a drive adapted to expel the drug from the reservoir;
a controller functionally coupled to the reader and the drive, the controller being programmed to:
(a) identify the drug contained in the reservoir based on the drug information,
(b) calculate or determine an acceptable range of injection times for the identified drug,
(c) determine a tailored injection time within the acceptable range of injection times, and
(d) set a delivery speed of the drive such that the drug is expelled from the reservoir over the tailored injection time.

2. The drug delivery system of claim 1, wherein the controller calculates or determines the acceptable range of injection times at least in part based on the drug information.

3. The drug delivery system of claim 2, wherein the drug information comprises at least one of viscosity, dose volume, minimum injection time to achieve drug efficacy, maximum injection time to achieve drug efficacy, minimum injection time to curtail injection pain, and maximum injection time to curtail injection pain.

4. The drug delivery system of claim 1,
wherein the controller includes a memory containing a drug information dataset having a list of possible drugs, each possible drug associated with drug information; and
wherein calculating or determining the acceptable range of injection times for the identified drug includes accessing the drug information dataset, determining that the identified drug is one possible drug in the list of possible drugs, and receiving the drug information associated with the identified drug in the drug information dataset.

5. The drug delivery system of claim 1,
wherein the drug delivery system further comprises an input device functionally coupled to the controller,
wherein the controller is further programmed to receive a preferred injection speed from the input device, and
wherein the tailored injection time is based on the preferred injection speed.

6. The drug delivery system of claim 5, wherein the input device includes two or more relative injection speed options, each relative injection speed option equal to a percentage of a longest possible injection time within the range of acceptable injection times, and wherein the preferred injection speed is the relative injection speed option selected using the input device.

7. The drug delivery system of claim 6, wherein the input device includes a touchscreen, and wherein the controller causes the input device to display the two or more relative injection speed options.

8. The drug delivery system of claim 6, wherein each of the two or more relative injection speed options are displayed as one of: a written adjective, the percentage of the longest possible injection, and a point on a sliding scale.

9. The drug delivery system of claim 6, wherein the two or more relative injection speeds are each unique physical buttons of the drug delivery device.

10. The drug delivery system of claim 1, wherein the identifier is an RFID tag or an NFC tag.

11. A method of preparing a drug delivery device for delivering a drug product, the method comprising:
(a) identifying a drug contained in a reservoir of a drug delivery system based on a reader of the drug delivery system reading an identifier on the reservoir;
(b) calculating an acceptable range of injection times for the identified drug;
(c) receiving a preferred injection speed from an input device of the drug delivery system;
(d) determining a tailored injection time within the acceptable range of injection times based on the preferred injection speed; and
(e) setting a delivery speed of a drive of the drug delivery system such that the drug is expelled from the reservoir over the tailored injection time.

12. The method of claim 11, wherein calculating the acceptable range of injection times for the identified drug includes receiving drug information from the identifier on the reservoir.

13. The method of claim 12, wherein the drug information includes at least one of viscosity, dose volume, minimum injection time to achieve drug efficacy, maximum injection time to achieve drug efficacy, minimum injection time to curtail injection pain, and maximum injection time to curtail injection pain.

14. The method of claim 11, wherein calculating the acceptable range of injection times for the identified drug includes accessing a drug information dataset provided in a memory of the controller, determining that the identified drug is one possible drug in a list of possible drugs, and receiving drug information associated with the identified drug in the drug information dataset.

15. The method of claim 12, wherein the drug information includes at least one of viscosity, dose volume, minimum injection time to achieve drug efficacy, maximum injection time to achieve drug efficacy, minimum injection time to curtail injection pain, and maximum injection time to curtail injection pain.

16. The method of claim 11, further comprising presenting two or more relative injection speed options, each relative injection speed option equal to a percentage of a longest possible injection time within the range of acceptable injection times, allowing selection of one of the two or more relative injection speed options, and using the relative injection speed option selected as the preferred injection speed.

17. The method of claim 16, further comprising causing the input device to display the two or more relative injection speed options on a touchscreen.

18. The method of claim 17, wherein each of the two or more relative injection speed options are displayed as one of: a written adjective, the percentage of the longest possible injection, and a point on a sliding scale.

19. The method of claim 16, wherein allowing selection of one of the two or more relative injection speed options includes associating the two or more relative injection speeds with unique physical buttons of the drug delivery device.

20. The method of claim 11, wherein reading an identifier on the reservoir comprises reading an RFID tag or an NFC tag.
